SEQUENCE LISTING (22 sequences)

SEQ ID NO: 1 - RNA, 74 nt, cocksfoot mottle virus
ugauaauagagcgaagaaagacacacuguuaucguuccccucccgaaucagagguugagaaguagcuuagaugu

SEQ ID NO: 2 - DNA, 68 bp, cocksfoot mottle virus
tgataatagtgcgaagaaagacacactgttatcgttcccctcccgaatcagaggttgagaagtagctt

SEQ ID NO: 3 - RNA, 40 nt, Alfalfa mosaic virus
guuuuuauuuuuaauuuucuuucaaauacuuccaucauga

SEQ ID NO: 4 - DNA, 40 bp, Alfalfa mosaic virus
gtttttatttttaattttctttcaaatacttccatcatga

SEQ ID NO: 5 - RNA, 42 nt, Potato virus X
  alpha sequence
gaaaacuaaaccauacaccaacaacacaaccaaacccaccac

SEQ ID NO: 6 - RNA, 46 nt, Potato virus X
  beta sequence
gcccaauuguuacacacccgcuuggaaaaguaagucuaacaaaugg

SEQ ID NO: 7 - DNA, 87 bp, Potato virus X
  alphabeta leader
gaaactaaaccatacaccaacaacacaaccaaacccaccacgcccaattgttacacacccgcttggaaaagtaagtctaacaaatgg

SEQ ID NO: 8 - RNA, 72 nt, Tobacco mosaic virus
guauuuuuacaacaauuaccaacaacaacaaacaacaaacaacauuacaauuacuauuuacaauuacaaugg

SEQ ID NO: 9 - DNA, 72 bp, Tobacco mosaic virus
gtatttttacaacaattaccaacaacaacaaacaacaaacaacattacaattactatttacaattacaatgg

SEQ ID NO: 10 - RNA, 8 nt, Artificial Sequence
  Description of Artificial Sequence Synthetic oligonucleotide
caccaugg

SEQ ID NO: 11 - DNA, 8 bp, Artificial Sequence
  Description of Combined DNA/RNA Molecule Synthetic oligonucleotide
ttccaugg

SEQ ID NO: 12 - RNA, 9 nt, Artificial Sequence
  Description of Artificial Sequence Synthetic oligonucleotide
aacaauggc

SEQ ID NO: 13 - RNA, 8 nt, Artificial Sequence
  Description of Artificial Sequence Synthetic oligonucleotide
accucgag

SEQ ID NO: 14 - DNA, 15 bp, Artificial Sequence
  Description of Artificial Sequence Synthetic oligonucleotide
aattcgagctcggta

SEQ ID NO: 15 - DNA, 82 bp, cocksfoot mottle virus
acctcgagtgataatagtgcgaagaaagacacactgttatcgttcccctcccgaatcagaggttgagaagtagcttccatgg

SEQ ID NO: 16 - DNA, 53 bp, Alfalfa mosaic virus
acctcgagtttttatttttaattttctttcaaatacttccatcatgaccatgg

SEQ ID NO: 17 - DNA, 109 bp, Potato virus X
acctcgagaattcgaaactaaaccatacaccaacaacacaaccaaacccaccacgcccaattgttacacacccgcttggaaaagtaagtctaacaaatggacaccatgg

SEQ ID NO: 18 - DNA, 88 bp, Tobacco mosaic virus
acctcgagtatttttacaacaattaccaacaacaacaaacaacaaacaacattacaattactatttacaattacaatggacaccatgg

SEQ ID NO: 19 - DNA, 29 bp, Artificial Sequence
  Description of Artificial Sequence Synthetic oligonucleotide
acctcgagaattcgagctcggtaccatgg

SEQ ID NO: 20 - DNA, 85 bp, cocksfoot mottle virus
ccggcctcgagtgataatagtgcgaagaaagacacactgttatcgttcccctcccgaatcagaggttgagaagtagcttccatgg

SEQ ID NO: 21 - DNA, 91 bp, Tobacco mosaic virus
ccggcctcgagtatttttacaacaattaccaacaacaacaaacaacaaacaacattacaattactatttacaattacaatggacaccatgg

SEQ ID NO: 22 - DNA, 32 bp, Artificial Sequence
  Description of Artificial Sequence Synthetic oligonucleotide
ccggcctcgagaattcgagctcggtaccatgg

(19) United States
(12) Patent Application Publication
(10) Pub. No.: US 2003/0167520 A1
(43) Pub. Date: Sep. 4, 2003

(54) Use of a nucleotide sequence for enhancing protein synthesis and expression of proteins

(76) Inventors: Teemu Teeri, Espoo (FI); Arno Kristian Aspegren, Helsinki (FI); Kristiina Maria Makinen, Klaukkala (FI); Mart Saarma, Helsinki (FI)

Correspondence Address: KUBOVCIK & KUBOVCIK, SUITE 710, 900 17TH STREET NW, WASHINGTON, DC, 20006

(21) Appl. No.: 10/182,257

(22) PCT Filed: Jan. 26, 2001
(86) PCT No.: PCT/FI01/00067

(30) Foreign Application Priority Data
Jan. 28, 2000 (FI) 20000182

(51) Int. Cl. A01H 1/00; C12N 15/82; A01H 5/00

(52) U.S. Cl. 800/279; 800/320.1; 800/320; 800/320.2; 800/320.3

(57) ABSTRACT

The present invention is related to the use of nucleotide sequences substantially similar to the cDNA sequence (SEQ ID NO:2:) obtainable from the leader sequence (SEQ ID NO:1:) of the Cocksfoot mottle virus (CfMV) which is capable of enhancing protein synthesis and expression of proteins, especially in plants such as cereals. Also disclosed is a method for producing potential enhancer elements by selecting 5′UTRs having a capacity of producing hairpin loop structures and preparing substantially similar nucleic acid sequences. In addition a method for enhancing the expression in plants as well as the properties characteristic for the nucleotide sequence which are responsible for the enhanced expression.

### THE TECHNICAL FIELD OF THE INVENTION

The present invention is related to the use of an isolated and purified nucleotide sequence substantially similar to (SEQ ID NO:2:) the leader sequence (SEQ ID NO:1:) of the Cocksfoot mottle virus (CfMV), which sequence is capable of enhancing protein synthesis or expression of a protein.

### THE BACKGROUND OF THE INVENTION

The 5′untranslated regions (5′UTRs) of many capped and uncapped RNAs of plant viruses are known to enhance the expression of chimeric genes in vitro and in vivo. Most of the in vivo studies have been made in cells of dicotyledonic (dicot) plants and using 5′UTRs from dicot-specific viruses, but the few results obtained in monocotyledonic (monocot) cells indicate differences in the compatibility of the 5′UTRs between dicots and monocots. The untranslated leader of tobacco mosaic virus RNA (TMV) acts as a translational enhancer in monocot plants, such as maize and rice protoplasts, but to a significally lower extent than in dicot systems, such as tobacco or carrot systems. The only 5′UTR capable of stimulating expression in both systems is the cauliflower mosaic virus (CaMV) 35S RNA leader. The only 5′UTR of a monocot-specific virus tested for its ability to enhance translation of chimeric RNAs in plant cells is the brome mosaic virus RNA3-leader, which does not confer any higher enhancement of gene expression than the leader sequence of tobacco mosaic virus (Gallie and Young, 1994).

The objectives of the present invention is to provide a nucleotide sequence capable of enhancing expression of genes and proteins, preferably in plants, including both gymnosperms and angiosperms as well as conifers and monocotyledons and dicotyledons, preferably industrially useful crop plants and especially in monocots such as cereals.

At the same time it is an objective of the present invention to provide methods for enhancing gene expression, the ultimate goal being improved protein production.

The objectives of the present invention are achieved by providing nucleotide sequences substantially similar with the 5′-terminus leader sequence of Cocksfoot mottle virus (SEQ ID NO:1:) and/or other substantially similar sequences capable of forming a stable hairpin structure by internal base pairing in the 5′-terminal end of the leader sequence.

### THE SUMMARY OF THE INVENTION

The characteristics of the present invention are as defined in the claims.

### A SHORT DESCRIPTION OF THE DRAWINGS

FIG. 1 depicts sequences of the 5′UTRs used in the comparative studies of the present invention.

FIG. 2 depicts marker gene constructs used in the present invention.

FIG. 3 depicts the constructs demonstrating enhanced expression of protein synthesis in tobacco.

FIG. 4 depicts the computer-predicted secondary structure of the 34 first 5-end nucleotides of native CfMVε RNA sequence (SEQ ID NO:1:).

FIG. 5 depicts the computer-predicted secondary RNA structure of a native chimeric 5′UTR.

### THE DETAILED DESCRIPTION OF THE INVENTION

Definitions

In the present invention the terms used have the meaning they generally have in the fields of conventional botany, plant breeding, plant virology, plant biochemistry and production of transgenic plants, including recombinant DNA technology as well as agriculture, horticulture and forestry. Some terms, however, are used with a somewhat deviating or broader meaning in this context. Accordingly, in order to avoid uncertainty caused by terms with unclear meaning some of the terms used in this specification and in the claims are defined in more detail below.

The term “plants” includes plant cells, plant tissues, plant organs as well as whole plants. The term includes both gymnosperms and/or angiosperms, including conifers, monocot and/or dicot plants as well as algae.

“Crop plants” mean plants of industrial importance, i.e. applicable in agriculture and forestry, and they especially include cereals, such as corn (maize), rice, wheat, oats and barley.

The plant viruses in the present invention include plant picornavirus-like viruses, particularly sobemoviruses, most particularly “Cocksfoot mottle virus (CfMV)” which is a member of the group sobemoviruses.

The term “nucleotide sequence” includes but is not restricted to double or single stranded DNA and/or RNA.

The term “fragments” means the smallest elements of the nucleotide sequence still contributing to the expression enhancement. The “fragments” can be effective alone but often their effect is additive. Accordingly, it is useful to combine one or more “fragments” in any order or direction forming “dimers” and/or “multimers” of “fragments” with the same or different sequences.

The term “enhancing expression” means improving or increasing expression including transcription, mRNA stabilization, RNA transportation, translation and protein stabilization, the ultimate goal being improved quantity of desired protein. Even if the preferred embodiment of the present invention is to provide enhanced protein synthesis in plants, the use of the nucleotide sequence of the present invention is not limited to plants. The nucleotide sequence of the present invention can be inserted in any eukaryotic or procaryotic transformation and/or expression vectors or DNA constructs compatible with and capable of transforming respective host organism.

The term “enhancer” or “enhancer element” means a nucleotide sequence component or a fragment of a nucleotide sequence having the capacity of increasing or improving expression as defined above.

The term “leader sequence” means a sequence in the beginning of messenger-RNA that is not necessarily translated to an amino acid sequence.

The term “transformed” means altered by adding and/or changing genetic information.

The term “method for selecting and preparing nucleotide sequences with an increased capability of enhancing expression” means the selection of certain 5′-terminal leader sequences in RNA plant viruses, which 5′-terminal leader sequences are “capable of forming a stable stem loop structure” as determined by secondary structure predicting computer programs. “Hairpin structure or stem loop” means a single stranded nucleotide sequence, e.g. a single stranded RNA capable of forming a hairpin loop structure by stable internal basepairing or by forming hydrogen bonds within the same strand. In this invention it especially means a structure substantially similar with that formed in the 5′-terminus of the leader sequence of Cocksfoot mottle virus.

The term “substantially similar” means a sequence having a homology of at least 60%, preferably 70%, more preferably 80%, most preferably 90%.

### THE GENERAL DESCRIPTION OF THE INVENTION

The present invention is related to a method for enhancing protein synthesis and/or expression of proteins. The enhancer is developed for increasing the expression of proteins in plants, particularly for increasing the protein synthesis in crop plants, such as cereals, representing monocotyledonic plants. As said above the use of the enhancer sequence is not limited to plants. The method is based on the use of a nucleotide sequence having the capacity of enhancing expression, i.e. enhancer elements. Such elements are with a high probability to be found for example from 5′UTRs of capped or uncapped RNAs in plant viruses. Said 5′-UTRs are selected by checking the 5′UTRs for the presence of nucleotide sequences capable of forming hairpin loop structures by stable internal base-pairing in their 5′-terminus.

Such UTRs can be used as models for preparing nucleotide sequences which are substantially similar with the selected 5′UTRs capable of forming hairpin loops in their 5′terminus. Said nucleotide sequences can be used in any transformation and/or expression vectors, plasmids or DNA constructs, in addition, to the plant vectors disclosed in the present invention, by functionally inserting them in said vectors. Crop plants, especially cereals can be transformed by per se known methods using said nucleotide sequences or said plant vectors comprising said nucleotide sequences.

The invention is based on results obtained in studies made on the leader sequence (SEQ ID NO:1:) of the Cocksfoot mottle virus, but said results are more broadly applicable for those skilled in the art.

As described above Cocksfoot mottle virus is a member of the sobemoviruses, i.e. plant RNA viruses. The genomic RNA of Cocksfoot mottle sobemovirus was isolated and characterized by Mäkinen et al. (1995). The genome of CfMV is a 4082 base-pairs long, plus-stranded RNA molecule.

For the initial studies a set of plant expression vectors were constructed utilizing cDNAs (SEQ ID NO:2:) for the CfMV leader sequence (CfMVε) (SEQ ID NO:1:). Nucleotide sequences, i.e. cDNA sequences obtainable from 5′UTRs of naturally capped RNAs from three dicot-specific viruses, including the leader sequence (SEQ ID NO:4:) of alfalfa mosaic virus (AMV) RNA4 (AMV5′) (Jobling and Gerke, 1987), the leader sequence (SEQ ID NO:9:) of tobacco mosaic virus (TMVΩ, Gallie & Young, 1994) and the leader α-sequence (SEQ ID NO:5:) and the β-sequence (SEQ ID NO:6:) of potato virus X RNA-leader (PVXαβ (SEQ ID NO:7:), Huisman et al., 1988) were used in comparative studies of expression enhancement.

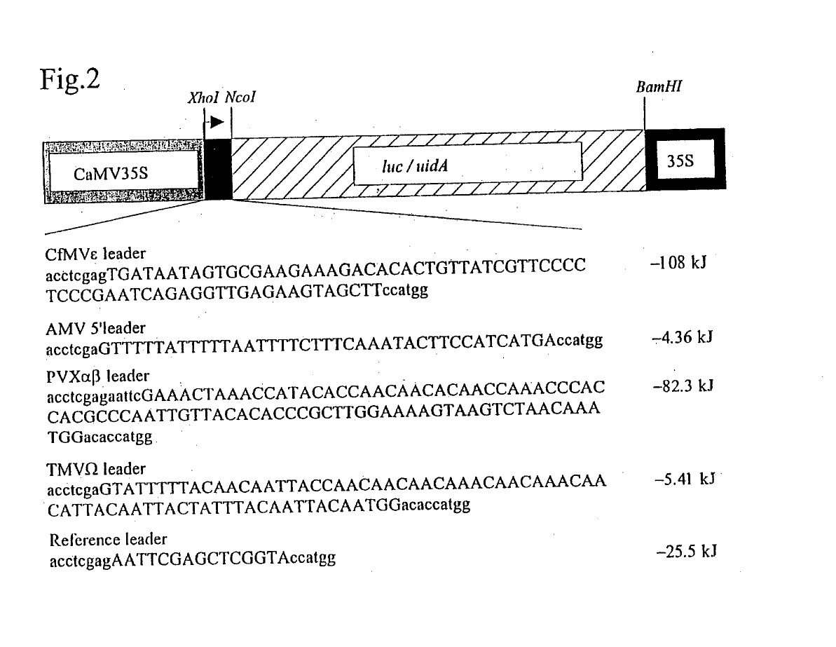

The translation initiation codon of all constructs used in the experiments of the present invention is located in the context of the Nco1 recognition sequence ⁻3 CACCAUGG (SEQ ID NO:10:) in AMV, PVXαβ, TMVΩ and TTCCAUGG (SEQ ID NO:11:) in CfMV. The consensus sequence for translation initiation in plants proposed by Lütcke et al. (1987) is ⁻3 AACAAUGGC (SEQ ID NO:12:). The ATG context of the leader sequences differed from the consensus: all at positions −1 and −4 (the A of ATG being +1), and the CfMVε-leader sequence (SEQ ID NO:2:) also at position −3 (FIG. 2).

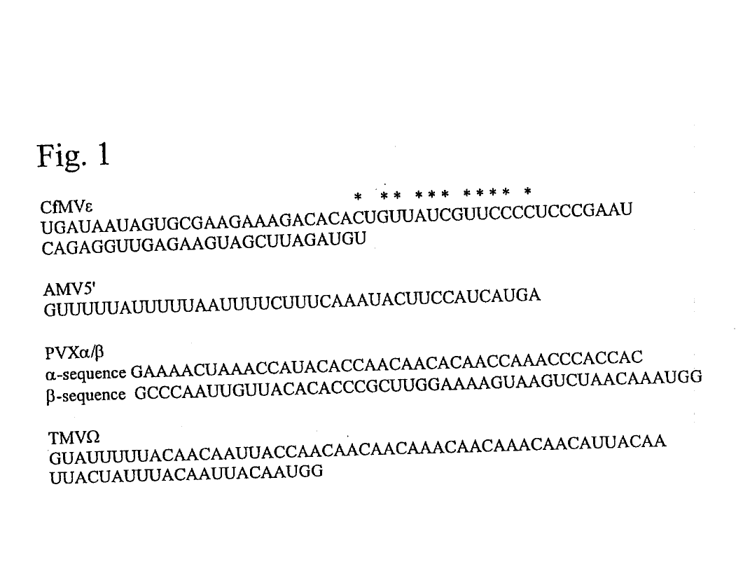

FIG. 1 shows the sequences of the 5′UTRs used in this study. Nucleotides with complementarity to the 18S ribosomal RNA (rRNA) consensus sequence according to Hagenbühle et al. (1978) are marked with asterisks on the sequence of CfMVε (SEQ ID NO:1). In FIG. 2 the cDNAs encoding different 5′UTRs (black box) were inserted downstream of the site for transcription initiation (arrow). The native sequence of the cDNA for each viral 5′UTR is marked in capital letters and vector derived sequences including the Xho1 and Nco1 sites used in vector construction are marked in lower case. The calculated stabilities (G in kJ/mol) of secondary structure within the 5′UTRs are shown on the right. In plant systems, the effect of the −3 position on translational efficiency seems not to be as significant as in animal systems (Koziel et al. 1996). In summary, it can be concluded that the differences of the initiation codon context between leader sequences do not cause any apparent differences in translational efficiency, at least not in favor of the CfMVε-leader sequence (SEQ ID NO:1:) and (SEQ ID NO:2:), and that the effects on gene expression must be explained by the properties of the leader sequences themselves.

The modification of proximal sequences of the elements and the addition of vector-derived nucleotides 5′ and 3′ of the viral leaders (FIG. 2) may have an impact on their function. However, studies in which steady state levels of mRNA and enzyme activity have been quantitated from transgenic plants expressing reporter genes fused to similar AMV5′and TMVΩ. derivatives (Datla et al., 1993 and Dowson Day et al., 1993), have shown that, although the insertion of heterologous 5′UTRs downstream of the CaMV 35S promoter cap site has effects on transcript levels, the major impact of the engineered leader sequences on the expression of their genes is exerted at translational level. Zelenina et al. (1992) showed in vitro, that also PVXαβ retains its translation enhancing properties despite a downstream vector-derived sequence and different spacer sequences preceeding reporter genes.

The cDNAs encoding the viral 5′UTRs were inserted into the polylinker region downstream of the putative CaMV 35S cap site (Guilley et al. 1982) in plant expression vectors and the effects of the different leader sequences on the expression of the reporter genes were analyzed on the level of enzyme activity. This is a versatile way for testing the regulatory elements, but does not distinguish between transcriptional and translational events controlling the expression of the engineered genes. Because AMV5′, TMVΩ and PVXαβ have all been shown to function as translational enhancers (Gallie, 1996 and wherein cited references), they are especially useful in comparative studies for determining, whether the expression capacity of the leader sequence of Cocksfoot mottle virus is enhanced.

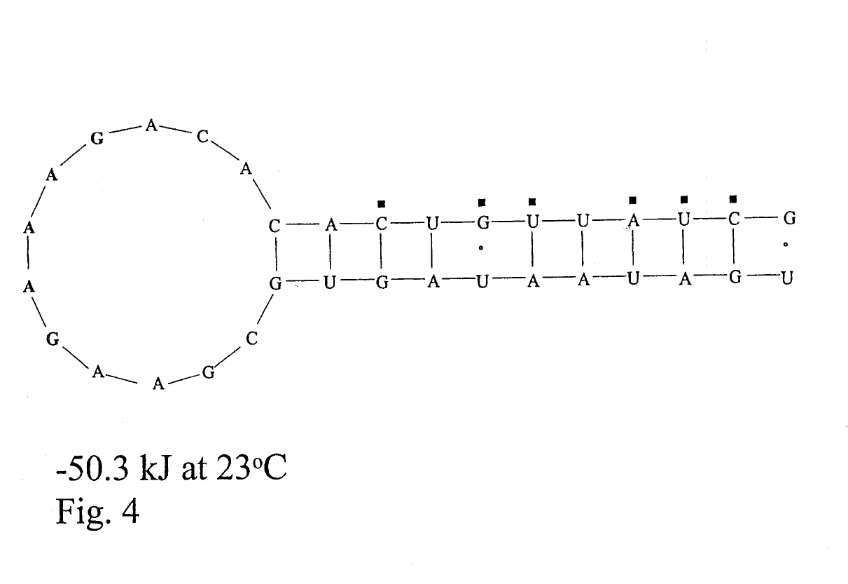

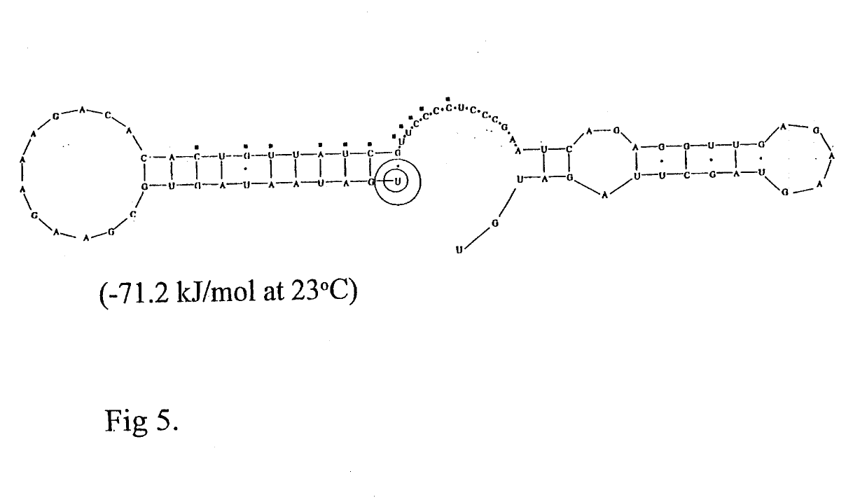

In the present invention computer-based folding predictions of leader sequences (Gehrke et al., 1983; Sleat et al., 1988) have been used to indicate, that in contrast to the predicted folds of other leader sequences used in this study, the CfMVε-leader has a potential to form a stable structure at the 5′-end. For Example, FIG. 4 depicts the predicted RNA secondary structure of the 34 first 5′-end nucleotides of CfMV and FIG. 5 shows the predicted RNA secondary structure of the native 5′UTR of CfMV. Note that intramolecular base pairing with the vector derived sequence 5′-ACCUCGAG-3′(SEQ ID NO:13:) increases the potential for formation of the hairpin structure at the 5′end of the leader when compared to the native CfMVε (SEQ ID NO:1:).

This stem-loop structure is maintained in RNA secondary structure predictions for the CfMVε-leader, when placed at the 5′-terminal end of luciferase (LUC) and β-glucuronidase (GUS) mRNAs (data not shown). This putative structure may be of significance in the cap-independent translation of CfMV RNA and other RNAs with an ε-leader. One possibility is, that the 5′structure is maintained during translation, but provides a suitable environment for internal entry for ribosomes downstream this structure.

Some of the translational enhancement conferred by 5′UTRs is believed to be sequence-dependent. The sequence of the leader sequences of TMVΩ and PVXαβ contain sequence motifs complementary to the 3′terminal sequence of 18S rRNA. A significant complementarity to the 3′terminus of 18S rRNA was also found in the CfMVε leader sequence (FIG. 1). This being a further evidence of the fact that the hairpin-loop forming capability might be a useful tool for developing new effective enhancer. The fact that a homologous structure is also found at the 5′end of RNAs of other sobemoviruses (Ryabov et al., 1996) is an indication that other nucleotide sequences useful as enhancers can be found among other sobemoviruses.

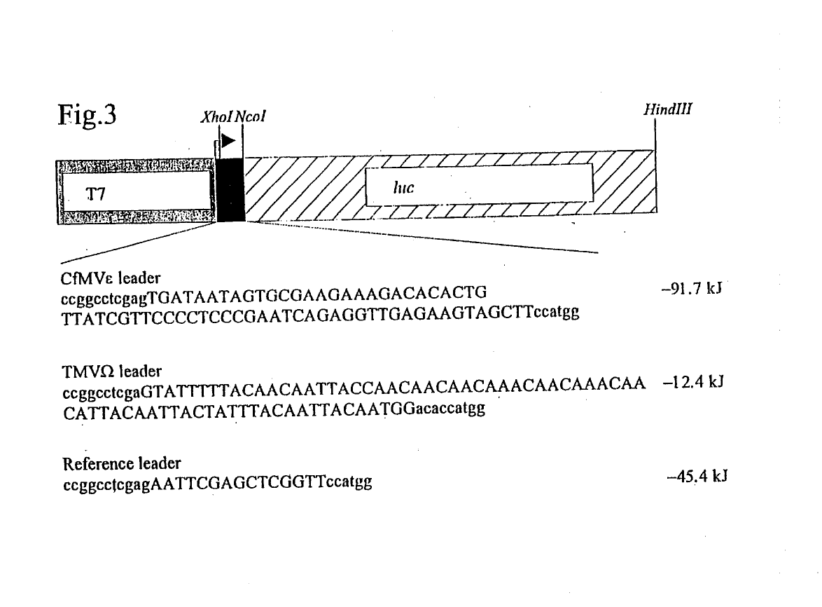

It was shown that the 5′UTR of Cocksfoot mottle virus RNA (CfMVε), when inserted into the untranslated leader of two different reporter genes, enhances expression of these genes two- to three-fold in tobacco protoplasts and suspension cultured barley cells. Three previously well characterized 5′UTRs from RNAs of dicot viruses; AMV5′, TMVΩ and PVXαβ, were used as references (FIG. 2). These elements conferred three- to five-fold enhancement in tobacco protoplasts, which is in good agreement with other transient expression studies performed with similar constructs (Datla et al., 1993, Dowson Day et al., 1993, Zelenina et al., 1992) (FIG. 3). However in contrast to CfMVε, all of these 5′UTRs failed to enhance gene expression in barley cells. In general, the viral leader sequences appeared to have a more stimulatory effect on the expression of luc than on uidA.

Furthermore, it was shown that CfMVε enhances transient expression of two reporter genes in tobacco protoplasts and especially in barley cells. The fact, that the 5′UTRs of the dicot specific viruses all fail to enhance gene expression in barley cells, provides additional evidence for differences on the function of leader sequences in dicots and monocots.

In the present invention it was shown that CfMVε belongs to those 5′UTRs of plant viruses, that enhance gene expression in plant cells. Computer analysis of the primary and secondary structure of CfMVε shows sequence and structure motifs with putative roles in cap-independent initiation of translation. In contrast to the 5′UTRs derived from the three dicot viruses, the CfMVε leader sequence enhances gene expression also in barley. Therefore, nucleotide sequence substantially similar with nucleotide sequence of CfMVε and/or forming similar hairpin structures can be used as models for the 5′UTR of choice, when constructing expression vectors for plants. Such elements ensure efficient expression of foreign genes not only in barley but also in other transgenic crop plants, including cereals.

In the present invention it was shown that the fundamental difference between monocots and dicots may exist in the degree to which the translational machinery can utilize specific 5′UTR of mRNA. The evolution of plant viruses may have led to specialized features of 5′UTRs, which enable efficient capture of the translational machinery in plants within the host range of particular viruses. In case of CfMVε, the identification of such putative monocot-specific features of leader sequences may be of importance both for the more detailed understanding of the function of 5′UTRs and genetic engineering of cereals.

The present invention is further described in the following part in which the methodology and results are described in detail. These methods as well as the results obtained should not be interpreted as restricting the scope of the protection. Based on said description those skilled in the art can easily think of developing other equally well functioning desirable and advantageous applications for agriculture and forestry.

Materials and methods

Plasmids

Genes coding for firefly luciferase (luc) and bacterial β-glucuronidase (uidA) were inserted in the polylinker region between the CaMV 35S promoter and terminator in a plant expression vector (plasmid) pRT101 (Töpfer et al., 1993). The resulting plant expression vectors encode an untranslated polylinker-derived leader sequence comprising 25 bp preceeding the start codon for translation (ATG contained in the Nco1 recognition site) of the reporter genes (FIG. 1). These 35S-luc and 35S-uidA vectors were used as references in transient expression studies. The cDNAs for viral 5′UTRs were obtained by polymerase chain reaction (PCR) amplification (CfMVε), annealing of oligonucleotides (AMV5′ and TMVΩ) or subcloning from plasmid (PVXαβ from pTZ-5X, a kind gift from J. Atabekov, (Zelenina, et al, 1999)). They were subcloned between the Xho1 and Nco1 sites in the untranslated leader of the 35S-luc and 35S-uidA vectors (FIG. 2), replacing most of the polylinker. Relevant portions between the promoter and the reporter genes were sequenced using the ALF DNA sequencer (Pharmacia LKB).

Large scale plasmid isolation was carried out according to the alkaline lysis method of Birnboim and Doly (1979). Plasmid preparations were further purified using Qiagen^{R}-columns and eluted into TE buffer (10 mM Tris and 1 mM EDTA, pH8). The DNA concentration in purified samples was determined spectrophotometrically and diluted to 1 mg/ml with TE buffer. To control experimental variation in transient expression experiments, each plasmid with one marker was mixed at a 1:1 concentration ratio with a plasmid expressing the second marker. For electroporation of tobacco protoplasts, the internal standards were pANU5 and pANU6 (35S-uidA and 35S-luc constructs with the reference leader, FIG. 1), and for bombardment of barley cells pAHC18 (ubiquitin-luc fusion, Christensen and Quail, 1996) and pHTT515 (ubiquitin-uidA fusion, subcloned from pAHC25, Christensen and Quail, 1996).

Particle bombardment of tissue cultured barley cells

Plasmid DNA was precipitated on tungsten particles and transferred to suspension cultured cells of barley (Hordeum vulgare L. cv. Pokko) using the Biolistic^{R} PDS-1000/He device. Particle bombardment and culture of the nonembrygenic P1 cells (VTT-G-93001) was performed essentially as described by Ritala et al. (1993). At least two independent experiments with five repetitions for each construct was performed.

Isolation and electroporation of tobacco protoplasts

Protoplasts were isolated from surface sterilized leaves of tobacco (Nicotiana tabacum) grown in greenhouse. Isolation, electroporation and culture of protoplasts was performed essentially as described by Suntio and Teeri (1994), except that the electroporations were done in 1 ml spectrophotometer cuvettes, using a pair of platinum plates 9 mm apart as electrodes, and giving the pulse from a 25 uF capacitor loaded to 550 V (BioRad Gene Pulser^{R}). Two independent electroporation experiments with duplicate DNA samples (10F μg per 2.5H×10⁶ protoplasts) were carried out to compare each set of constructs.

Analysis of transient expression

LUC and GUS enzyme activities were measured from samples 34-38 h after gene transfer. Soluble protein was extracted from protoplast pellets and collected barley cells in 1.5 ml microcentrifuge tubes on ice by brief grinding with a plastic pestle (Kontes) in cold cell lysis buffer no.2 (Bio-Orbit, Turku, Finland). Cell debris was removed from lysates by two centrifugations (20000 g, 5 min.). Protein concentration in cleared supernatants was determined using the Bio-Rad protein assay kit (Catalog 500-0006). GUS activity was determined by the fluorometric assay described by Jefferson (1987). For LUC assays, 10 μl aliquots of lysate was added to 100 μl reaction buffer (Luciferase Assay System, Promega), and relative luminiscense in the reaction was measured with a luminometer (Bio-Orbit, model 1253). Specific activities for GUS and LUC were determined per mg protein. Normalized values for enzyme activity were determined by dividing values for GUS or LUC activity with the value of the internal standard in the particular sample. Data from experiments comparing each set of plasmids were calculated as a percentage of the mean activity for the 35S-luc and 35S-uidA constructs with the reference leader (100%), and significant differences were tested for by a t-test and variance analysis (anova).

Computer analysis of leader sequences

The secondary structures of all leader sequences used were predicted by using the RNA-DRAW program (http://broccoli-mfn.ki.se/rnadraw/rnadraw.html). The cell energy calculations for the stability of the secondary structures were performed at the same temperature where the actual in vivo expression experiments were done.

RESULTS

The cDNAs encoding the 5′UTRs of RNAs of viruses TMVΩ and the cocksfoot mottle virus RNA leader (CfMVε) were inserted into the untranslated leader sequences luc reporter gene in plant expression vectors (FIG. 2). To determine the expression enhancement LUC expression levels were measured by translation experiments.

In in vitro translation experiments as shown in Table 1 no significant enhancement of luciferase (LUC) mRNA translation was demonstrated, when LUC was fused to the CfMVε-leader (FIG. 3). The same constructs were used to produce transcripts to determine the relative enhancement of protein synthesis in tobacco protoplasts (Table 1).

The cDNAs encoding the 5′UTRs of RNAs of three dicot specific viruses (AMV5′, TMVΩ and PVXαβ) and the cocksfoot mottle virus RNA leader (CfMVε) were inserted into the untranslated leader sequences of uidA and luc reporter genes in plant expression vectors (FIG. 2). To determine the effects of the different 5′UTRs on GUS and LUC expression levels, the constructs were transferred to tobacco protoplasts by electroporation and to the suspension cultured barley cells by particle bombardment.

Transient expression from constructs harboring viral leader sequences were compared to the control uidA and luc constructs with a 25 bp leader (FIG. 2), thus giving an estimate for the enhancement conferred by the viral 5′UTRs. In tobacco protoplasts, all 5′UTRs enhanced expression of the reporter genes: AMV5′and CfMVε two- to three-fold, TMVΩ and PVXαβ four- to five-fold (Table 2), respectively. In barley cells, however, only CfMVε appeared to have a stimulatory effect on uidA and luc expression. The relative level of gene expression conferred by this monocot specific virus-derived 5′UTR was three times higher than that for PVXαβ, the only dicot virus-derived 5′UTR able to confer comparable expression levels for the reporter genes as the reference leader (Table 2).

Primary and secondary structure analysis of leader sequences

The secondary structure predictions of the leaders used for the in vivo experiments (FIG. 1) proposed remarkable differences in their folding potential. The reference leader, the AMV RNA4-leader and TMVΩ have low potential for stable intramolecular base-pairing. The significantly higher ΔG value for the structure of the PVXαβ-leader is due to a putative hairpin structure formed by base-pairing within the β-sequence, while the 5′-proximal α-sequence is unstructured (Smirnyagina et al. 1991). In contrast to the other leaders CfMVε has high potential to form a stable structure at the 5′-end (FIG. 2A). This stem-loop structure is formed by 34 first nucleotides of the leader, which free energy is −50.3 kJ/mol, while the free energy of the complete leader is −71.2 kJ/mol at 23° C. (FIG. 4 and 5). In the chimeric constructs used in this study, basepairing between vector derived sequences and the CfMV sequence increases the length of the 5′ stem structure by three basepairs and raises the free energy of the complete leader to −99.1 kJ/mol. The 5′-terminal structure partly overlaps with a region, nucleotides 25-39, in CfMVε that is complementary with the consensus sequence of 18S ribosomal RNA 3′ termini derived from several organisms (Hagenbühle et al. 1978, Wu et al., 1987). Ten out of thirteen nucleotides of this region are able to basepaire with the most 3′ proximal nucleotides of this consensus sequence (4 G-C, 5 A-U and 1 G-U pairs, FIG. 1). Similar complementarity to 18S rRNA is also present in the 5′UTR of the RNA of another sobernovirus, the Southern bean mosaic virus (SBMV). In SBMV the complementarity is 10 out of 15 nucleotides (Wu et al., 1987). In this study the expression studies were done in tobacco and barley systems. The complementarity of tobacco 18S rRNAs (accession number X59789) and CfMVε is 11 out of 15 nucleotides while in barley (Azad and Deacon, 1980) it is 7 out of 12 nucleotides.

TABLE 1
Relative enhancement of protein synthesis conferred by the viral UTRs in
tobacco protoplasts. LUC values are presented as means relative to con-
structs with the 25 bp reference leader.

| Element | Proportional LUC |
|---|---|
| | 1.00 −+ 0.09 |
| TMVΩ | 3.62 −+ 0.32 |
| CFMVE | 1.46 −+ 0.13 |

Table 2.

Relative enhancement of protein expression conferred by the viral 5′UTRs in tobacco protoplasts and barley cells with cDNA constructs. LUC and GUS values are presented as means relative to constructs with the 25 bp reference leader.

| | TOBACCO | | BARLEY | |
|---|---|---|---|---|
| | LUC | GUS | LUC | GUS |
| REF | 1.00 ± 0.50 | 1.00 ± 0.09 | 1.00 ± 0.58 | 1.00 ± 0.28 |
| AMV5′ | 3.22 ± 1.59 | 2.85 ± 0.50 | 0.36 ± 0.22 | 0.33 ± 0.09 |
| TMVΩ, | 5.22 ± 1.88 | 4.85 ± 1.80 | 0.44 ± 0.36 | 0.06 ± 0.01 |
| PVXαβ | 5.22 ± 2.82 | 3.95 ± 0.77 | 1.06 ± 0.57 | 0.60 ± 0.11 |
| CfMVε | 3.03 ± 1.06 | 2.40 ± 0.99 | 3.43 ± 1.00 | 1.74 ± 0.47 |

REFERENCES

Azad, A. A. & Deacon, N. J. (1980) Nucleic Acids Res. 8, 4365-4376.

Birnboim, H. C. & Doly, J. (1979) Nucl Acids Res 7: 1513-1523.

Christensen, A. H. & Quail, P. H. (1996) Transgenic Res 5: 213-218.

Datla, R. S. S., et al. (1993) Plant Sci 94: 139-149.

Dowson Day, M., et al. (1993) Plant Mol Biol 23: 97-109.

Hagenbüchle, O., et al. (1978) Cell 13, 551-563.

Gallie, D. R., et al. (1987) Nucl Acids Res 15: 8693-8711.

Gallie, D. R., et al. (1989) Plant Cell 1: 301-311.

Gallie, D. R. & Walbot, V. (1992) Nucl Acids Res 20: 4631-4638.

Gallie, D. R. & Young, T. E. (1994) Plant Physiol 106: 929-939.

Gallie, D. R. (1996) Plant Mol Biol 32: 145-158.

Gehrke, L., et al. (1983) Biochemistry 22: 5157-5164.

Ghosh, A., et al. (1979) Nucl Acids Research 7: 2137-2146.

Guilley, H., et al. (1982) Cell 30: 763-773.

Huisman, M. J., et al. (1988) J Gen Virol 69: 1789-1798.

Jefferson, R. A. (1987) Plant Mol Biol Rep 5: 387-405.

Jobling, S. A. & Gehrke, L. (1987) Nature 325: 622-625.

Kozak, M. (1989) J Cell Biol 108: 229-241.

Koziel, M. G., et al. (1996) Plant Mol Biol 32: 393-405.

Leathers, V., et al. (1993) Mol Cell Biol 13: 5331-5347.

Lütcke, H. A., et al. (1987) EMBO J 6: 43-48.

Mandeles, S. (1968) J Biol Chem 243: 3671-3674.

McElroy, D. & Brettel, R. I. S. (1994) Tibtech 12: 62-68.

Mäkinen, K., et al. (1995) J Gen Virol 76: 2817-2825.

Ow, D. W., et al. (1986) Science 234: 856-859.

Pooggin, M. M. & Skrjabin, K. G. (1992) Mol Gen Genet 234: 329-331.

Ritala, A., et al. (1993) Plant Cell Rep 12: 435-440.

Ryabov, E. V., et al. (1996) Mol Plant Path 86: 391-397.

Shine, J. & Dalgarno, L. (1974) Proc Nat Acad Sci 71: 1342-1346.

Sleat, D. E., et al. (1988) Eur J Biochem 175: 75-86.

Smirnyagina, E. V., et al. (1991) Biochimie 73: 587-598.

Sleat, D. E., et al. (1992) In: Genetic Engineering with Plant viruses (Wilson, T. M. A. and Davies, J. W., eds.). Boca Raton, Fla.: CRC Press, pp. 55-113.

Suntio, T. M. & Teeri, T. H. (1994) Plant Mol Biol Rep 12: 43-57.

Truve, E., et al.(1997) Arch Phytopath Pflanz 30: 473485.

Töpfer, R., et al. (1993) Method Enzymol 217: 66-78.

Wu, S., et al. (1987) Virology 161, 73-80.

Zelenina, D. A., et al. (1992) FEBS lett 296 (3): 267-270.

We claim:

1. Use of a nucleotide sequence as an enhancer for improving protein synthesis in monocotyledonous plants, characterized in, that said nucleotide sequence is substantially identical with the nucleotide sequence SEQ ID NO:2 and/or fragments and/or multimers thereof and said nucleotide sequence is obtainable from the leader sequence (SEQ ID NO:1) of Cocksfoot mottle virus (CfMV) and capable of enhancing the protein synthesis and enhancing the expression of proteins.

2. Use of the nucleotide sequence according to claim 1, characterized in, that the plant in which the expression is enhanced is a cereal.

3. Use of the nucleotide sequence according to claim 1, characterized in, that the plant in which the expression is enhanced is selected from a group consisting of corn (maize), rice, wheat, oats and barley.

4. Use of the nucleotide sequence according to claim 1, characterized in, that the plant in which the expression is enhanced is barley.

5. Use of the nucleotide sequence according to claim 1 for preparing vector constructs.

6. Use of the nucleotide sequence according to claim 1 for transforming host organisms.
